(19) United States
(12) Patent Application Publication
(10) Pub. No.: US 2025/0169703 A1
(43) Pub. Date: May 29, 2025

(54) METHOD AND DEVICE FOR OBTAINING VASCULAR PRESSURE DIFFERENCE

(71) Applicant: SHANGHAI PULSE MEDICAL TECHNOLOGY, INC., Shanghai City (CN)

(72) Inventors: SHENGXIAN TU, Shanghai City (CN); WEI YU, Shanghai City (CN); SHUZHAN CHEN, Shanghai City (CN); YINGGUANG LI, Shanghai City (CN)

(21) Appl. No.: 19/026,262

(22) Filed: Jan. 16, 2025

Related U.S. Application Data
(63) Continuation of application No. 17/254,255, filed on Dec. 18, 2020.

(30) Foreign Application Priority Data
Jun. 20, 2018 (CN) 201810637750.8

(51) Int. Cl. A61B 5/021; A61B 5/00; A61B 5/02; A61B 5/0285; G16H 50/50

(52) U.S. Cl. CPC A61B 5/021; A61B 5/02007; A61B 5/0285; A61B 5/4887; G16H 50/50

(57) ABSTRACT

The present invention provides a method and device for obtaining the pressure difference of blood vessels. The method for obtaining the pressure difference of a blood vessel includes: receiving anatomical data of a part of a blood vessel segment, obtaining a geometric model of a target blood vessel according to the anatomical data; obtaining a blood flow model of the target blood vessel and the target blood vessel according to the anatomical data and combining individual data The blood flow velocity V; the geometric model is preprocessed, the cross-sectional morphological model is established, and the shape difference function f(x) of the target blood vessel lumen is calculated, based on the shape difference function f(x) of the target blood vessel lumen And the blood flow velocity V, the pressure difference value ΔP at any two positions of the target blood vessel is calculated. The method for obtaining vascular pressure difference provided by the present invention introduces the concept of morphology to clarify the influence of vascular morphology on the calculation of vascular pressure difference and improve the accuracy of calculation of vascular pressure difference.

### CROSS-REFERENCE TO RELATED APPLICATIONS

This application is a Continuation application of U.S. patent application Ser. No. 17/254,255, filed Dec. 18, 2020, and entitled “METHOD AND DEVICE FOR OBTAINING VASCULAR PRESSURE DIFFERENCE”, which is a 35 U.S.C. § 371 National Phase conversion of International (PCT) Patent Application No. PCT/CN2018/109077, filed on Sep. 30, 2018, which claims benefit of Chinese patent application No. 201810637750.8, filed on Jun. 20, 2018, the contents of which are hereby incorporated by reference in their entirety herein.

### BACKGROUND

### 1. Technical Field

The invention relates to a method and a device for obtaining vascular pressure difference, belonging to the medical technical field.

### 2. Description of Related Art

The deposition of lipids and carbohydrates in human blood on the vascular wall will form plaques on the vascular wall, which will then cause vascular stenosis; especially the vascular stenosis that occurs near the coronary artery of the heart will cause insufficient blood supply to the myocardium and induce coronary heart disease Diseases such as angina and angina pectoris pose a serious threat to human health. According to statistics, there are currently about 11 million patients with coronary heart disease in my country, and the number of patients undergoing cardiovascular interventional surgery is increasing by more than 10% every year.

Although conventional medical testing methods such as coronary angiography and CT can show the severity of coronary artery stenosis, they cannot accurately evaluate coronary ischemia. In order to improve the accuracy of coronary vascular function evaluation, in 1993 Pijls proposed a new indicator of coronary vascular function through pressure measurement-Fractional Flow Reserve (FFR). After long-term basic and clinical research, it has become the gold standard for functional evaluation of coronary stenosis.

Fractional blood flow reserve (FFR) usually refers to the myocardial blood flow reserve, which is defined as the ratio of the maximum blood flow that the diseased coronary artery can provide to the myocardium to the maximum blood flow when the coronary artery is completely normal. Studies have shown that the maximum congestion in the coronary artery in the state that the ratio of blood flow can be replaced by pressure. That is, the measurement of FFR value can be calculated by measuring the pressure at the distal end of coronary artery stenosis and the pressure at the proximal end of coronary artery stenosis through the pressure sensor under the state of maximum coronary hyperemia. In recent years, the method of measuring the FFR value based on the pressure guide wire has gradually entered clinical application and has become an effective method for patients with coronary heart disease to obtain accurate diagnosis; however, the pressure guide wire is likely to cause damage to the patient's blood vessel during the intervention process; at the same time, through pressure The measurement of the FFR value by the guide wire requires the injection of adenosine/ATP and other drugs to ensure that the coronary artery reaches the maximum congestion state. Some patients will feel uncomfortable due to the injection of the drug, which makes the method of measuring the FFR value based on the pressure guide wire have greater limitations. In addition, although the measurement of FFR guided by the pressure guide wire is an important indicator of coronary stenosis hemodynamics, the high cost of the pressure guide wire and the difficulty in the operation of the interventional vascular process have severely restricted the measurement of FFR value based on the pressure guide wire. The promotion and use of methods.

With the development of CT and three-dimensional angiography reconstruction technology and the popularization and application of 3D coronary artery geometric reconstruction technology in the field of hemodynamics, at the same time, in order to reduce the harm to the human body and the measurement cost during the FFR value measurement process, medical imaging FFR calculation technology has become a research focus.

In the prior art, Taylor et al. applied computer fluid dynamics to computed tomography coronary angiography (CTA), using CTA to obtain coronary anatomical data, including the volume and quality of the myocardium supplied by the blood vessel, to estimate the maximum coronary blood flow, Simulate the resistance of the microcirculation downstream of the blood vessel, and solve the fluid equation as the boundary condition of the computational fluid dynamics simulation, and obtain the non-invasive method FFRCT to calculate the FFR.

In fact, although the prior art provides methods for determining the fractional flow reserve (FFR) from different angles and methods, the essence is to pass the blood flow pressure Pa at the proximal end of the target vessel and the proximal end of the target vessel. FFR is calculated by the difference ΔP between the blood flow pressure at the endpoint and the distal endpoint. In the actual process of blood flow, that is, during the actual calculation of the blood flow pressure difference ΔP, the location, size and type of the lesion will all affect the calculation of the blood flow pressure difference ΔP; Different medical history information and physiological characteristics will also affect the difference ΔP of blood flow pressure; therefore, in the prior art, the FFR calculated by the difference ΔP of blood flow pressure often deviates from the actual value. The results of FFR evaluation of coronary stenosis function have errors.

Hence, there is a need to provide a new method of obtaining vascular pressure difference to solve the problems.

### SUMMARY

The objective of the present invention is to provide a method for obtaining the pressure difference of blood vessels to at least solve one of the technical problems existing in the prior art. The method for obtaining the vascular pressure difference provided by the present invention introduces the concept of morphology to clarify the influence of plaque information on the calculation of the vascular pressure difference and improve the accuracy of the calculation of the vascular pressure difference.

In order achieve the above-mentioned object of the invention, the present invention provides a method for obtaining a blood vessel pressure difference, and the method for obtaining a blood vessel pressure difference includes:
Receiving anatomical data of the blood vessel, and obtaining a geometric model of the target blood vessel according to the anatomical data;
Acquire a blood flow model of the target blood vessel according to the anatomical data combined with individual data, and obtain the blood flow velocity V of the target blood vessel according to the blood flow model;
Preprocessing the geometric model to establish a cross-sectional shape model of the target blood vessel at various positions between the proximal end and the distal end;
Using the proximal end point of the target blood vessel as a reference point, the cross-sectional morphological model at different scales is fitted to calculate the morphological difference function f(x) of the target vessel lumen, and the scale is the calculated morphological difference function f(x) is the distance between two adjacent cross sections;
Based on the morphological difference function f(x) of the target vessel lumen and the blood flow velocity V, the pressure difference value ΔP at any two positions of the target vessel is calculated.

As an improvement of the present invention, wherein the blood vessels include coronary blood vessels, branch blood vessels from coronary blood vessels, vascular trees and single blood vessel segments; the individual data include individual general parameters and individual specific parameters; the blood flow model includes at least the blood flow velocity V of the target.

As an improvement of the present invention, wherein the pressure difference value ΔP is calculated by calculating the morphological difference function f(x) of the target vessel lumen at different scales and the blood flow model of the target vessel, the calculation formula of the ΔP at different scales is:

$$Δ P = ( {c}_{1} V + {c}_{2} {V}^{2} + … + {c}_{m} {V}^{m} ) * [ {α}_{1} * \int {f}_{1} ( x ) d x + {α}_{2} * \int {f}_{2} ( x ) dx + … + {α}_{n} * \int {f}_{n} ( x ) d x ]$$

Among them, V is the blood flow velocity, which is obtained directly/indirectly through the blood flow model;
c₁, c₂, . . . , cₘ respectively denote the parameter coefficients of blood flow velocity V;
α₁, α₂, . . . , αₙ are the weighting coefficients of the morphological difference functions ƒ₁(x), ƒ₂(x), . . . , ƒₙ(x) of the vascular lumen at different scales;
m is a natural number greater than or equal to 1;
n is a natural number whose scale is greater than or equal to 1;
The different scales include a first scale, a second scale, . . . , an n-th scale;
The first-scale morphological difference function ƒ₁(x) is used to detect the geometric morphological difference between two adjacent cross-sectional morphological models caused by the first type of lesion feature;
The second-scale morphological difference function ƒ₂(x) is used to detect the geometric morphological difference between two adjacent cross-sectional morphological models caused by the second type of lesion feature;
The n-th scale morphological difference function ƒₙ(x) is used to detect the geometric morphological difference between two adjacent cross-sectional morphological models caused by the n-th lesion feature; wherein, n is a natural number greater than or equal to 1.

As an improvement of the present invention, wherein the establishment of the cross-sectional morphological model comprises:
S1. Define the cross section at the proximal end of the target blood vessel as a reference plane, and obtain the center diameter of the geometric model through a centerline extraction and establishment method;
S2. Establish a coordinate system with the center point of the reference surface as the origin, segment the target blood vessel in a direction perpendicular to the center diameter line, and project the inner and outer edges of each cross section in the coordinate system to obtain the target, the plane geometric image of the cross-section of the lumen at each position of the blood vessel, the establishment of the cross-sectional morphological model is completed.

As an improvement of the present invention, wherein the cross-sectional morphological model includes the presence or absence of plaques on each cross-section, the position of the plaque, the size of the plaque, and the angle formed by the plaque, plaque composition and changes in plaque composition, changes in plaque shape and plaque shape.

As an improvement of the present invention, wherein the morphological difference function f(x) is used to indicate that the cross-sectional morphological changes at different positions of the target blood vessel follow the distance x from the position to the reference point. Function of change;

The acquisition of the morphological difference function f(x) includes:
Based on the cross-sectional morphological model, establish the shape function of each cross-section; the shape function includes area function, diameter function and edge position function;
Fit the morphological functions of two adjacent cross sections, and obtain the difference change function of two adjacent cross sections at different scales;

Take the proximal end of the target vessel as the reference point, obtain the rate of change of the lumen shape with the distance x from the reference point according to the difference change function, and normalize the position parameters of the target vessel from the proximal end to the distal end Processing to obtain the morphological difference function f(x).

As an improvement of the present invention, wherein the obtaining of the blood flow model further comprises correcting the blood flow model through medical history information and/or physiological parameter information, and passing the corrected the blood flow model is obtained; the blood flow model includes a fixed blood flow model and a personalized blood flow model;

The personalized blood flow model includes a resting state blood flow model and a load state blood flow model; when the blood flow model is a resting state blood flow model, the blood flow velocity V can pass the speed at which intravascular fluid is filled Obtained by calculation; or calculated by the shape of the vascular tree;

The morphology of the vascular tree includes at least one or more of the area and volume of the vascular tree and the lumen diameter of the vascular segment in the vascular tree; when the blood flow velocity V is obtained by calculating the shape of the vascular tree, the geometric parameters also include one or more of the length, perfusion area, and branch angle of the vessel segment in the vessel tree.

As an improvement of the present invention, wherein the blood flow velocity V includes the blood flow velocity of the target blood vessel in the maximum congestion state and the blood flow velocity in the resting state; or, the preprocessing of the geometric model includes the correction of the geometric model through medical history information and/or physiological parameter information.

In order achieve the above-mentioned object of the invention, the present invention provides a device for obtaining blood vessel pressure difference, characterized in that it comprises:
A data collector, which is used to obtain and store the geometric parameters of the target blood vessel in the anatomical model of the vascular system;
A pressure difference processor, which is used to establish a blood flow model of the target blood vessel, and a geometric model corresponding to the target blood vessel established based on the geometric parameters;
The pressure difference processor is further configured to correct the geometric model and/or blood flow model, and obtain a cross-sectional shape model and a blood vessel pressure difference calculation model based on the corrected geometric model and the blood flow model; at the same time, according to the blood vessel pressure difference calculation model and hemodynamics, the pressure difference value ΔP of the target blood vessel is obtained.

As an improvement of the present invention, wherein the geometric model is obtained by measuring and calculating the image data of the anatomical model and fitting and calibrating; and the cross-sectional morphological model is obtained by The geometric model is directly/indirectly obtained; or, the cross-sectional morphological model includes the presence or absence of patches on each cross-section, the location of the patches, the size of the patches, the angle formed by the patches, the composition of the patches, and the patches changes in composition, plaque shape and changes in plaque shape.

As an improvement of the present invention, wherein the geometric model obtained by the pressure difference processor includes at least one vascular tree, and the vascular tree includes at least a segment of aorta or at least a segment of aorta and Multiple coronary arteries originating from the aorta; or the geometric model includes at least a single vessel segment.

As an improvement of the present invention, wherein the apparatus for obtaining blood vessel pressure difference further comprises a speed collector, and the speed collector is used to obtain the blood flow speed of the target blood vessel, and the blood flow the speed is used to calculate the pressure difference value ΔP between the proximal end and the distal end of the target blood vessel;

The speed collector includes a speed calculation module and a speed extraction module; the speed extraction module can directly collect the blood flow speed through the data collector, or directly extract the blood flow speed through the blood flow model;

The speed calculation module includes a speed conversion module and a speed measurement module. The blood flow speed can be obtained by converting the speed of fluid filling in blood vessels by the speed conversion module, and can also be obtained by converting the shape of the blood vessel tree in the geometric model by the speed. Obtained by calculation module.

In order achieve the above-mentioned object of the invention, the present invention provides a device for obtaining blood flow reserve score, characterized in that it comprises:
A data collector, which is used to obtain and store the geometric parameters of the target blood vessel in the anatomical model of the blood vessel device;
A blood flow information processor, the blood flow information processor being used to establish a blood flow model of the target blood vessel, and to establish a geometric model corresponding to the target blood vessel based on the geometric parameters;
The blood flow information processor is also used to correct the geometric model and the blood flow model to obtain a cross-sectional shape model, and obtain a vascular pressure difference calculation model based on the cross-sectional morphological model and the blood flow model and the maximum blood flow velocity of the target vessel; and according to the vascular pressure difference calculation model and the maximum blood flow velocity, combined with hemodynamics, the blood flow reserve fraction FFR is calculated and obtained.

As an improvement of the present invention, wherein the geometric model is obtained by measuring and calculating the image data of the anatomical model and fitting calibration; the cross-sectional morphological model is obtained by the geometric model is directly/converted to obtain;

When the image data received by the data collector is angiographic image data of the target blood vessel, the image data collected by the data collector is not less than two groups, and there is a collection angle between any two groups of the image data Difference, and the acquisition angle difference is not less than 20 degrees.

As an improvement of the present invention, wherein the cross-sectional morphological model includes the presence or absence of plaques on each cross-section, the position of the plaque, the size of the plaque, and the plaque the angle formed, the composition of the plaque and the change of the plaque composition, the shape of the plaque and the change of the shape of the plaque.

As an improvement of the present invention, wherein the geometric model obtained by the blood flow information processor includes at least one vascular tree, and the vascular tree includes at least a segment of aorta or at least a segment of aorta. Arteries and multiple coronary arteries originating from the aorta; or the geometric model includes at least a single vessel segment;

The blood flow model established by the blood flow information processor includes a fixed blood flow model and a personalized blood flow model; the personalized blood flow model includes a resting blood flow model and a stress blood flow model;

When the blood flow model is a resting blood flow model, the maximum blood flow velocity can be obtained by calculating the speed of fluid filling in the blood vessel; or by calculating the shape of the vascular tree;

The shape of the vascular tree includes at least one or more of the area and volume of the vascular tree and the lumen diameter of the vascular segment in the vascular tree; when the maximum blood flow velocity is obtained by calculating the shape of the vascular tree, the geometric parameters also include one or more of the length, perfusion area, and branch angle of the vessel segment in the vessel tree.

As an improvement of the present invention, wherein the device for obtaining blood flow reserve score further comprises a speed collector, which is used to obtain the maximum blood flow speed of the target blood vessel, and the maximum blood flow velocity is used to calculate the first blood flow pressure Pa at the proximal end of the target blood vessel and the pressure difference value ΔP between the proximal end and the distal end of the target blood vessel.

In order achieve the above-mentioned object of the invention, the present invention provides a device for obtaining a patient's blood vessel pressure difference, the device having a processor, characterized in that: the processor is configured to make the device execute the following steps:
Collect the anatomical data of the patient's blood vessel to be examined;
Establishing a blood vessel model of the patient's blood vessel to be examined according to the anatomical data;
Based on the blood vessel model, further establishing a lumen morphology model at different scales;
According to the preset morphological difference function, the vascular pressure difference between any two positions of the blood vessel to be examined is determined based on the lumen morphological model and the blood vessel model.

As an improvement of the present invention, wherein the scale is the distance between two adjacent cross sections;

The morphological difference function is obtained by fitting and establishing the lumen morphological model, and is used to represent the function of the cross-sectional morphological changes at different positions of the target blood vessel as the distance x from the position to the reference point changes; and the morphological difference function It includes a difference function related to the cross-sectional area or diameter or edge distance of the target blood vessel.

In order achieve the above-mentioned object of the invention, the present invention provides a method for obtaining vascular pressure difference, characterized in that the method includes:
Receiving anatomical data of the blood vessel, and obtaining a geometric model of the target blood vessel according to the anatomical data;
Preprocessing the geometric model to establish a cross-sectional shape model of the target blood vessel at various positions between the proximal end and the distal end;
Using the proximal end point of the target blood vessel as a reference point, the cross-sectional morphological model at different scales is fitted to calculate the morphological difference function f(x) of the target vessel lumen, and the scale is the calculated morphological difference function f(x) is the distance between two adjacent cross sections;
The calculation formula of the pressure difference value ΔP at any two positions of the target blood vessel at different scales is: ΔP=k*[α₁*∫ƒ₁(x)dx+α₂*∫ƒ₂(x)dx+ . . . +αₙ*∫ƒₙ(x)dx]
Among them, k is a correction parameter, and k is a constant greater than or equal to 1;
The α₁, α₂, . . . , αₙ are the weighting coefficients of the morphological difference functions ƒ₁(x), ƒ₂(x), . . . , ƒₙ(x) of the vascular lumen at different scales;
The different scales include a first scale, a second scale, . . . , an n-th scale;
The first-scale morphological difference function ƒ₁(x) is used to detect the geometric morphological difference between two adjacent cross-sectional morphological models caused by the first type of lesion feature;
The second-scale morphological difference function ƒ₂(x) is used to detect the geometric morphological difference between two adjacent cross-sectional morphological models caused by the second type of lesion feature;
The n-th scale morphological difference function ƒₙ(x) is used to detect the geometric morphological difference between two adjacent cross-sectional morphological models caused by the n-th lesion feature; wherein, n is a natural number greater than or equal to 1.

As an improvement of the present invention, wherein the correction parameter k is a value obtained directly/indirectly based on individual information;

The morphological difference function f(x) is used to represent the function of the cross-sectional morphological changes at different positions of the target blood vessel as the distance x from the position to the reference point changes.

The beneficial effects of the present invention are: the method for obtaining blood vessel pressure difference of the present invention obtains a plane geometric image at each cross-sectional position of the target blood vessel by establishing a cross-sectional shape model, and establishes a shape difference function by fitting the cross-sectional morphological model at different positions. In the process of calculating the pressure difference, the concept of cross-sectional shape is introduced, and the influence of factors such as the position and shape of the plaque in the lumen on the calculation of the vascular pressure difference is comprehensively considered; so that the calculation obtained by the method for obtaining the vascular pressure difference of the present invention The value of the blood vessel pressure difference is more accurate and can accurately reflect the pressure changes at both ends of the target blood vessel; it is ensured that the blood vessel pressure difference calculated by the method of the present invention is accurate and reliable when applied to the calculation of other blood flow characteristic values.

### BRIEF DESCRIPTION OF THE DRAWINGS

FIG. 1 is a schematic diagram of a geometric model of a target blood vessel of the present invention.

FIG. 2 is a schematic structural diagram of a cross-sectional morphological model at position D1 in FIG. 1.

FIG. 3 is a schematic structural diagram of a cross-sectional morphological model at position D2 in FIG. 1.

FIG. 4 is a schematic diagram of the structure after fitting the cross-sectional morphological model at positions D1 and D2 in FIG. 2 and FIG. 3.

FIG. 5 is a schematic diagram of a geometric model of the target blood vessel in another form of the present invention.

FIG. 6 is a schematic structural diagram of a cross-sectional morphological model at position D1 in FIG. 5.

FIG. 7 is a schematic diagram of the structure of the cross-sectional morphological model at the position D2 in FIG. 5.

FIG. 8 is a schematic diagram of the structure after fitting the cross-sectional morphological model at positions D1 and D2 in FIGS. 6 and 7.

FIG. 9 is a structural block diagram of an apparatus for obtaining a blood vessel pressure difference according to the present invention.

FIG. 10 is a structural block diagram of a device for obtaining a blood flow reserve score according to the present invention.

### DETAILED DESCRIPTION OF THE ILLUSTRATED EMBODIMENT

Reference will now be made to the drawing figures to describe the embodiments of the present disclosure in detail. In the following description, the same drawing reference numerals are used for the same elements in different drawings.

The present invention provides a method for obtaining vascular pressure difference, and the method for obtaining vascular pressure difference includes the following steps:
Receiving anatomical data of the blood vessel, and obtaining a geometric model of the target blood vessel according to the anatomical data;
Obtain a blood flow model of the target blood vessel according to the anatomical data combined with individual data;
Preprocessing the geometric model to establish a cross-sectional shape model of the target blood vessel at various positions between the proximal end and the distal end;
Using the proximal end point of the target blood vessel as a reference point, the cross-sectional morphological model at different scales is fitted to calculate the morphological difference function f(x) of the target vessel lumen, and the scale is the calculated morphological difference function f(x) is the distance between two adjacent cross sections;
Based on the morphological difference function f(x) of the target vessel lumen and the blood flow velocity V, the pressure difference value ΔP at any two positions of the target vessel is calculated.

Among them, the blood vessels include coronary blood vessels, branch blood vessels from coronary blood vessels, blood vessel trees and single blood vessel segments; the individual data includes individual general parameters and individual specific parameters.

Further, the pressure difference value ΔP is calculated through the morphological difference function f(x) at different scales and the blood flow model of the target blood vessel, and the calculation formula of the pressure difference value ΔP at different scales is: ΔP=(c₁V+c₂V²+ . . . +cₘV^{m})*[α₁*∫ƒ₁(x)dx+α₂*∫ƒ₂(x)dx+ . . . +αₙ*∫ƒₙ(x)dx]

Among them, V is the blood flow velocity, which is obtained directly/indirectly through the blood flow model;

The c₁ c₂, . . . , cₘ respectively denote the parameter coefficients of blood flow velocity V, which include multiple parameter coefficients such as blood viscosity influencing factors, blood turbulence influencing factors, and viscosity coefficient; further, m is a natural number greater than or equal to 1, to denote different parameters respectively The influence of the coefficient on the blood flow velocity V is to correct the pressure difference value ΔP to ensure the accuracy of the calculation of the pressure difference value ΔP. Preferably, in the present invention, the value of n is 2, and when m is 2, c1 is a parameter coefficient caused by blood flow friction, and c2 is a parameter coefficient caused by blood turbulence.

The α₁, α₂, . . . , αₙ are the weighting coefficients of the vascular lumens at different scales ƒ₁(x), ƒ₂(x), . . . , ƒₙ(x), where n is a natural number with a scale greater than or equal to 1; further, the increase of the weighting coefficient can further affect the morphological difference function ƒ(x) Make corrections to ensure the accuracy of the calculation of the morphological difference between the two cross sections.

Specifically, the different scales include a first scale, a second scale, . . . , an n-th scale;

The first-scale morphological difference function ƒ₁(x) is used to detect the geometric morphological difference between two adjacent cross-sectional morphological models caused by the first type of lesion feature;

The second-scale morphological difference function ƒ₂(x) is used to detect the geometric morphological difference between two adjacent cross-sectional morphological models caused by the second type of lesion feature;

The n-th scale morphological difference function ƒₙ(x) is used to detect the geometric morphological difference between two adjacent cross-sectional morphological models caused by the n-th lesion feature.

The cross-sectional morphological model is obtained directly/indirectly through the geometric model, and in the present invention, the geometric model includes at least geometric parameters such as the shape, diameter, and area of the target blood vessel. Further, the geometric parameters are also Including the bending angle of the blood vessel segment and other parameters that can reflect the actual shape of the target blood vessel. Specifically, the establishment of the cross-sectional morphological model includes the following steps:
S1. Define the cross section at the proximal end of the target blood vessel as a reference plane, and obtain the center diameter of the geometric model through the method of centerline extraction and establishment;
S2. Establish a coordinate system with the center point of the reference surface as the origin, segment the target blood vessel in a direction perpendicular to the center diameter line, and project the inner and outer edges of each cross section in the coordinate system to obtain the target plane geometric image of the cross-section of the lumen at each position of the blood vessel, and the establishment of the cross-sectional morphological model is completed.

Among them, the cross-sectional morphology model includes plaque information at each cross-sectional position, and the plaque information is the lesion information of the target blood vessel, and a large amount of data indicates that when the length of the plaque (i.e., the lesion) is >20 mm, Will cause the target blood vessel pressure difference value ΔP to increase, and further lead to errors in the calculation of blood flow characteristic values such as blood flow reserve fraction FFR; and when the composition of the plaque at the same cross section is complicated or the size is too large, the target blood vessel will be narrowed High rate, it will further lead to the increase of the target blood vessel pressure difference value ΔP; at the same time, when the plaque is in different positions, the myocardial volume area supplied by the target blood vessel is different, which will lead to the difference between the lesion position and the non-lesion position. The change of the ratio further affects the blood flow velocity V, which leads to deviation of the target blood vessel pressure difference value ΔP.

Therefore, when building the cross-sectional morphological model, the patch information also needs to include the presence or absence of the patch, the location of the patch, the size of the patch, the angle formed by the patch, the composition of the patch, and the composition of the patch. The change of the plaque shape and the change of the plaque shape, and in the present invention, the plane geometrical image of the lumen cross-section at each position needs to be referenced to the coordinate system established in step S2 to clarify each cross-section the location of the patch to facilitate subsequent fitting of the cross-sectional morphological model.

It should be noted that, in the process of establishing the cross-sectional morphology model, when the anatomical data is acquired by CT, OCT, IVUS and other detection methods, the cross-sectional morphology model can be directly acquired through the geometric model. It is only necessary to ensure that the origin and coordinate directions of each of the cross-sectional morphological models are consistent; when the anatomical data is acquired by X-ray and other detection means, since the geometric model is a three-dimensional model extending along the direction of blood flow, When the cross-sectional morphological model is established through the geometric model, the geometric model needs to be coordinated to accurately reflect the cross-sectional shape of each cross-section.

The method for obtaining the vascular pressure difference further includes fitting the cross-sectional morphological model at different scales, and calculating the morphological difference function f(x) of the target blood vessel lumen. Wherein, the morphological difference function f(x) is used to denote the cross-sectional morphological change at different positions of the target blood vessel as a function of the distance x from the position to the reference point; and the acquisition of the morphological difference function f(x) include:
Based on the cross-sectional morphological model, establish the shape function of each cross-section;
Fit the morphological functions of two adjacent cross sections, and obtain the difference change function of two adjacent cross sections at different scales;
Taking the proximal end of the target vessel as the reference point, obtain the rate of change of the lumen shape with the distance x from the reference point according to the difference change function, and normalize the position parameters of the target vessel from the proximal end to the distal end to obtain the shape difference function f(x) finally.

The morphological function includes an area function, a diameter function, or an edge distance function, that is, in the present invention, the difference between two adjacent cross sections at different scales can be obtained by fitting between the cross-sectional area, diameter, or edge distance function. Change function; further, the change rate of the lumen shape with the distance x from the reference point is obtained through the difference change function, and the shape difference function f(x) is obtained.

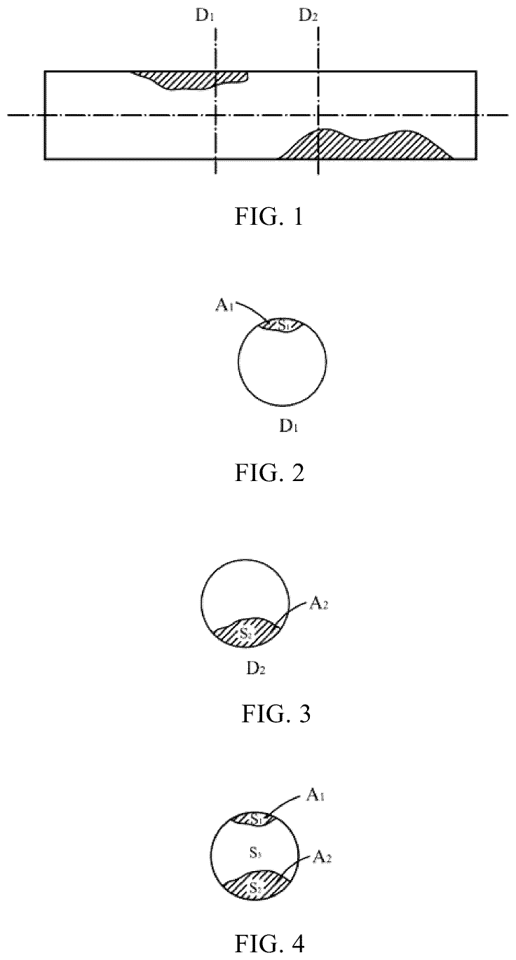

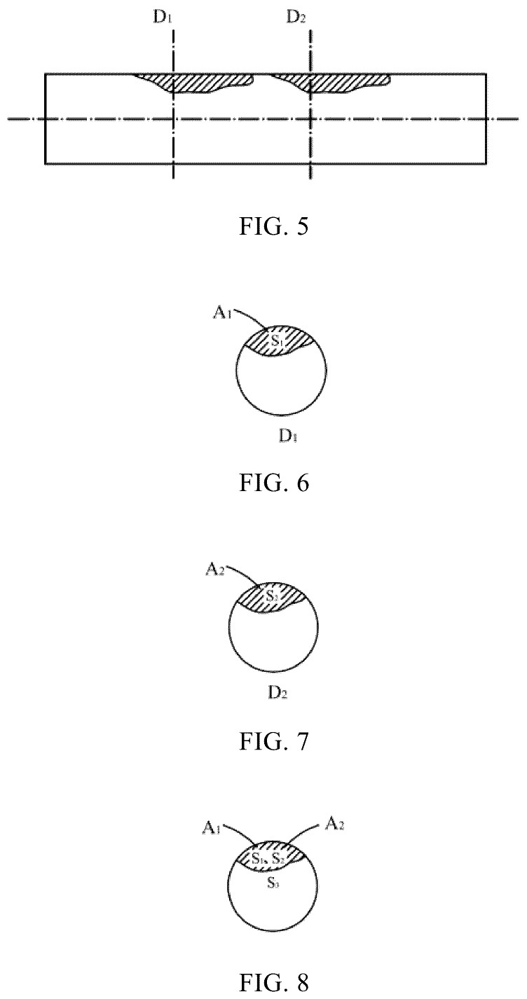

Specifically, when the morphological function is an area function, as shown in FIGS. 1 to 4, the two cross-sectional morphological models at positions D₁ and D₂ are fitted, and the cross-sectional morphological models at D₁ and D₂ are fitted after fitting, the area with increased vascular lumen plaque is A₁, corresponding to area S1; the area with reduced blood vessel lumen is A₂, corresponding to area S2. Since the vascular lumens (plaques) at the positions D₁ and D₂ do not overlap, when blood flows through D₁ to D₂, the blood pressure will change accordingly; at this time, the difference change function is the vascular tube The ratio of the area between the non-overlapping area (S1, S2) and the overlapping area (S3) in the cavity, or the ratio of the area of the non-overlapping area (S1, S2) to the total area (S1, S2, S3); and at this time, The morphological difference function f(x)>0, that is, there is a pressure difference between the cross sections D₁ and D₂. Further, when the vascular lumens (plaques) at the D₁ and D₂ positions completely overlap, as shown in FIGS. 5 to 8, the regions A₁ and A₂ completely overlap, that is, the areas of the non-overlapping regions A₁ and A₂ S1=S2=0. At this time, the difference change function is 0, that is, the morphological difference function f(x)=0. At this time, there is no pressure difference between the cross sections D₁ and D₂.

When the morphological function is a distance function, at this time, establish the correspondence between each point on the selected first lumen boundary and each point on the second lumen boundary, and then calculate each point on the first lumen boundary the distance between each point and each point on the boundary of the second lumen is subtracted from the distance along the center diameter of the blood vessel, and the sum or the average distance of all points is obtained. Specifically, if the distance from the corresponding point of the first lumen boundary and the second lumen boundary to the center meridian is both y, then the morphology of the first lumen and the second lumen are completely consistent, that is, the morphological difference function f (x)=0; if the distance from the corresponding point of the first lumen boundary and the second lumen boundary to the center meridian is different, the morphology of the first lumen and the second lumen are not completely consistent, that is, the morphological difference function f(x)>0.

Further, in the present invention, the calculation of the pressure difference value ΔP is also related to the blood flow velocity V of the target vessel, and in the present invention, the blood flow velocity V can be obtained directly through the blood flow model. Indirect acquisition.

Specifically, the blood flow model in the present invention includes a fixed blood flow model and a personalized blood flow model, and the blood flow model may be a data calculation model or a three-dimensional fluid flow model. The fixed blood flow model is an empirical blood flow model. When the blood flow model is a fixed blood flow model, the blood flow velocity V can be directly obtained from the fixed blood flow model, and is described in the present invention. The blood flow velocity V may also be a fixed parameter; it should be noted that the acquisition of the fixed blood flow model is directly established through the method of big data collection and simulation based on actual clinical experience.

The personalized blood flow model includes a resting state blood flow model and a load state blood flow model; when the blood flow model is a resting state blood flow model, the blood flow velocity V can be obtained by calculating the velocity of fluid filling In an embodiment of the present invention, the resting state blood flow model is a contrast agent blood flow model, and at this time, the blood flow velocity V is the target blood vessel obtained by using the gray-scale time fitting function for contrast during the imaging The average flow velocity of the contrast agent; or the average flow velocity of the contrast agent in the target blood vessel calculated by using the TIMI number frame method during the angiography process.

When the resting blood flow model is a CT blood flow model, the blood flow velocity V can be obtained by calculating the shape of the vascular tree, and the shape of the vascular tree includes at least the area, volume, and vascular tree One or more of the lumen diameters of the middle vascular segment; when the blood flow velocity V is obtained by calculating the shape of the vascular tree, the geometric parameters also include the length of the vascular segment in the vascular tree and the perfusion area And one or more of the branch angles.

In another embodiment of the present invention, the blood flow model is a stress blood flow model, at this time, the blood flow velocity V is the blood flow velocity V after the adenosine injection vessel is fully expanded, and at this time, the blood flow velocity V is the maximum blood flow velocity Vmax.

In particular, the blood flow velocity V in the present invention includes the blood flow velocity Vmax when the target blood vessel is in the maximum congestion state and the blood flow velocity Vqc in the resting state. When the target blood vessel is located in the coronary artery region, the blood flow velocity V is the blood flow velocity Vmax in the maximum congestion state. The further blood flow velocity Vmax can be obtained directly through the blood flow model, or obtained by converting the blood flow velocity V calculated by the blood flow model; when the target blood vessel is located in the peripheral vascular system, the blood flow velocity V is the blood flow velocity Vqc in the resting state.

It should be noted that, in order to ensure that the pressure difference value ΔP obtained by the method for obtaining vascular pressure difference of the present invention is accurate, the cross-sectional morphological model and the blood flow velocity are obtained through the geometric model and the blood flow model. At V, the blood flow model and/or the geometric model need to be corrected through medical history information and/or physiological parameter information, and in the present invention, the medical history information includes circulatory diseases that affect blood flow speed or blood viscosity, Respiratory system disease, nervous system disease, bone disease, digestive system disease, metabolic disease, family history, etc.; the physiological parameters include age, gender, blood pressure, body mass index, coronary artery dominant type and other directly obtainable physiological information.

Further, the factors affecting the pressure difference value ΔP also include myocardial microcirculation resistance (IMR) and whether there is collateral circulation. Specifically, when the target blood vessel has myocardial microcirculation resistance, it will affect the microcirculation perfusion, and further affect the blood flow velocity V of the target blood vessel, causing the blood flow velocity V to decrease, resulting in a decrease in the target blood vessel pressure difference value ΔP, which leads to There are errors in the calculation of blood flow characteristic values such as the blood flow reserve fraction FFR. When the target blood vessel has collateral circulation, it will cause the maximum blood flow through the target blood vessel to decrease, so that the target blood vessel pressure difference value ΔP decreases and the calculated value of the blood flow reserve fraction FFR increases.

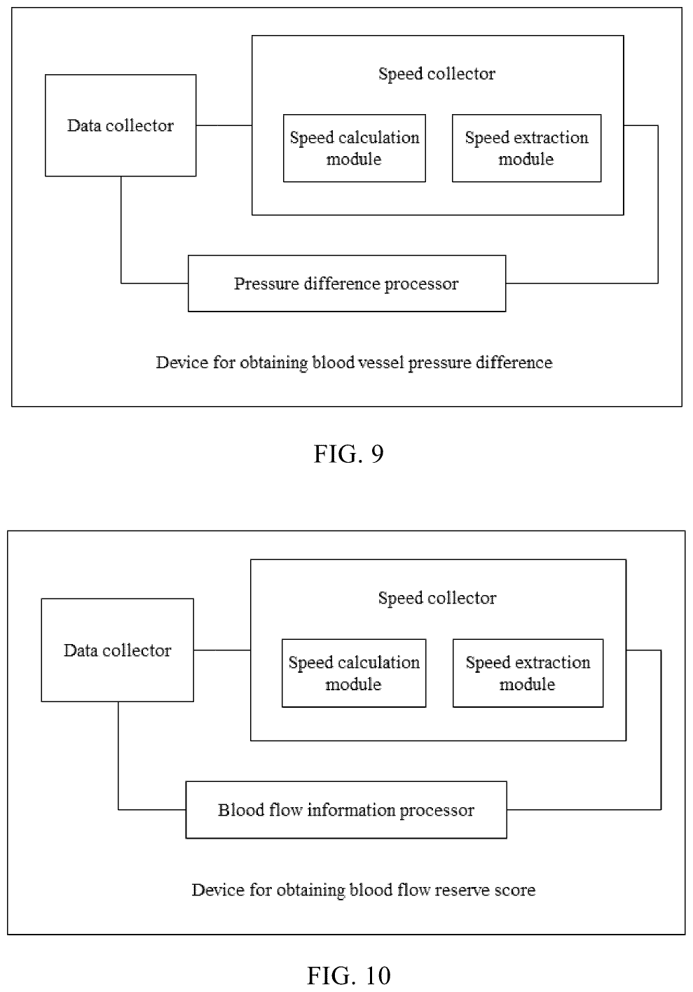

Please refer to FIG. 9, the present invention also provides a device for obtaining vascular pressure difference, and the device for obtaining vascular pressure difference includes:

A data collector, which is used to obtain and store the geometric parameters of the target blood vessel in the anatomical model of the vascular system;

A pressure difference processor, the pressure difference processor being used to establish a blood flow model of the target blood vessel, and a geometric model corresponding to the target blood vessel established based on the geometric parameters;

The pressure difference processor is further configured to correct the geometric model and/or blood flow model, and obtain a cross-sectional shape model and a blood vessel pressure difference calculation model based on the corrected geometric model and the blood flow model; At the same time, according to the vascular pressure difference calculation model and hemodynamics, the first blood flow pressure Pa at the proximal end of the target blood vessel and the pressure difference value ΔP between the proximal and distal end of the target blood vessel are obtained.

Further, the geometric model is obtained by measuring and calculating the image data of the anatomical model and fitting calibration; specifically, the geometric model obtained by the pressure difference processor includes at least the shape and diameter of the target blood vessel Geometric parameters such as area and area, the geometric parameters also include the bending angle of the blood vessel segment and other parameters that can reflect the actual shape of the target blood vessel; that is, in the present invention, the geometric model can be a single blood vessel segment or a blood vessel tree, and The vascular tree includes an aorta and a plurality of coronary arteries from the aorta.

The cross-sectional morphological model is obtained directly/indirectly through the geometric model, and the cross-sectional morphological model includes the presence or absence of plaques on each cross-section, the position of the plaque, the size of the plaque, and the formation of the plaque Angle, plaque composition and changes in plaque composition, plaque shape and changes in plaque shape.

Further, the apparatus for obtaining the pressure difference of a blood vessel further includes a speed collector, the speed collector is used to obtain the blood flow speed of the target blood vessel, and the blood flow speed is used to calculate the first blood flow pressure Pa at the end point of the proximal end of the target blood vessel and the pressure difference value ΔP between the proximal and distal end of the target vessel.

The speed collector includes a speed calculation module and a speed extraction module; the speed extraction module can directly collect the blood flow speed through the data collector, or directly extract the blood flow speed through the blood flow model.

The speed calculation module includes a speed conversion module and a speed measurement module. The blood flow speed can be obtained by converting the speed of fluid filling in blood vessels by the speed conversion module, and can also be obtained by the shape of the blood vessel tree in the geometric model by the speed obtained by calculation module.

Preferably, the calculation formula of the pressure difference value ΔP is: ΔP=(c₁V+c₂V²+ . . . +cₘV^{m})*[α₁*∫ƒ₁(x)dx+α₂*∫ƒ₂ (x)dx+ . . . +αₙ*∫ƒₙ(x)dx]

Among them, V is the blood flow velocity, which is obtained directly/indirectly through the blood flow model; the c₁, c₂, . . . , cₘ r denotes the parameter coefficients of blood flow velocity respectively, the parameter coefficients include multiple parameter coefficients such as blood viscosity influence factor, blood turbulence influence factor and viscosity coefficient; further, m is a natural number greater than or equal to 1, to denote different parameter coefficients respectively For the influence of blood flow velocity, the pressure difference value ΔP is corrected to ensure the accuracy of the calculation of the pressure difference value ΔP. Preferably, the value of n in the present invention is 2, and when m is 2, c₁ is a parameter coefficient caused by blood flow friction, and c₂ is a parameter coefficient caused by blood turbulence.

The α₁, α₂, . . . , αₙ are the weighting coefficients of the vascular lumens at different scales ƒ₁(x), ƒ₂(x), . . . , ƒₙ(x), where n is a natural number with a scale greater than or equal to 1; further, the increase of the weighting coefficient can further affect the morphological difference function ƒ(x) Make corrections to ensure the accuracy of the calculation of the morphological difference between the two cross sections.

Please refer to FIG. 10, the present invention also provides a device for obtaining a blood flow reserve fraction, the device for obtaining a blood flow reserve fraction includes:
A data collector, which is used to obtain and store the geometric parameters of the target blood vessel in the anatomical model of the blood vessel device;
A blood flow information processor, the blood flow information processor is used to establish a blood flow model of a target blood vessel, and establish a geometric model corresponding to the target blood vessel based on the geometric parameters;
The blood flow information processor is also used to modify the geometric model and the blood flow model to obtain a cross-sectional shape model, and to obtain a vascular pressure difference based on the cross-sectional morphological model and the blood flow model Calculate the model and the maximum blood flow velocity of the target vessel; calculate and obtain the blood flow reserve fraction FFR according to the calculation model of the vascular pressure difference and the maximum blood flow velocity in combination with hemodynamics.

The geometric model is obtained by the blood flow information processor by measuring the image data of the anatomical model acquired by the data collector and fitting and calibrating it; specifically, when the image data of the anatomical model is When acquired by CT, OCT, IVUS and other equipment, the data collector can directly collect the image data and transfer it to the blood flow information processor for fitting to establish a geometric model; and when the anatomical model is when the image data is acquired by a radiography method, when the data collector collects the image data, the image data is not less than two groups, and there is a difference in the acquisition angle between any two groups of the image data, and The difference in the acquisition angle is not less than 20 degrees. With such a setting, the geometric model obtained by the blood flow information processor can ensure that the geometric model is accurately established.

Further, the cross-sectional morphological model is obtained through direct/conversion of the geometric model, and the cross-sectional morphological model includes the presence or absence of patches on each cross-section, the location of the patches, the size of the patches, the angle formed by the plaque, the composition of the plaque and the change of the plaque composition, the shape of the plaque and the change of the plaque shape.

The blood flow model established by the blood flow information processor includes a fixed blood flow model and a personalized blood flow model; the personalized blood flow model includes a resting blood flow model and a stress blood flow model.

When the blood flow model is a resting blood flow model, the maximum blood flow velocity can be obtained by calculating the speed of fluid filling in the blood vessel; or by calculating the shape of the vascular tree. When the maximum blood flow velocity is obtained by calculating the shape of the vascular tree, the geometric model includes at least one vascular tree, and the vascular tree includes at least one aortic vessel segment or at least one aorta and Multiple coronary arteries, or the geometric model includes at least one single vessel segment; in this case, the geometric parameters also include one or more of the length of the vessel segment in the vascular tree, the perfusion area, and the branch angle The morphology of the vascular tree includes at least one or more of the area and volume of the vascular tree and the lumen diameter of the vascular segment in the vascular tree.

Further, the device for acquiring the blood vessel pressure difference further includes a velocity collector, the velocity collector is used to obtain the maximum blood flow velocity of the target blood vessel, and the maximum blood flow velocity is used to calculate the first blood flow pressure Pa at the proximal end of the target blood vessel, and the pressure difference value ΔP between the proximal and distal end of the target vessel.

Preferably, the calculation formula of the pressure difference value ΔP is: ΔP=(c₁V+c₂V²+ . . . +cₘV^{m})*[α₁*∫ƒ₁(x)dx+α₂*∫ƒ₂(x)dx+ . . . +αₙ*∫ƒₙ(x)dx]

Among them, the c₁, c₂, . . . , cₘ denotes the parameter coefficients of blood flow velocity respectively, the parameter coefficients include multiple parameter coefficients such as blood viscosity influence factor, blood turbulence influence factor and viscosity coefficient; further, m is a natural number greater than or equal to 1, to denote different parameter coefficients respectively For the influence of blood flow velocity, the pressure difference value ΔP is corrected to ensure the accuracy of the calculation of the pressure difference value ΔP. Preferably, the value of n in the present invention is 2, and when m is 2, c₁ is a parameter coefficient caused by blood flow friction, and c₂ is a parameter coefficient caused by blood turbulence.

The α₁, α₂, . . . , αₙ are the weighting coefficients of the vascular lumens at different scales ƒ₁(x), ƒ₂(x), . . . , ƒₙ(x), where n is a natural number with a scale greater than or equal to 1; further, the increase of the weighting coefficient can further affect the morphological difference function ƒ(x) Make corrections to ensure the accuracy of the calculation of the morphological difference between the two cross sections.

The present invention also provides a device for obtaining a patient's blood vessel pressure difference, the device having a processor, wherein the processor is configured to cause the device to perform the following steps:
Collect the anatomical data of the patient's blood vessel to be examined;
Establishing a blood vessel model of the patient's blood vessel to be examined according to the anatomical data;
Based on the blood vessel model, further establishing a lumen morphology model at different scales;
According to the preset morphological difference function, the vascular pressure difference between any two positions of the blood vessel to be examined is determined based on the lumen morphological model and the blood vessel model.

The “processor” includes any device that receives and/or generates signals, and the data processed by the processor can be text messages, instructions for object/fluid movement, input from applications, or some other information; the blood vessel to be inspected Alternative terms for can be target blood vessels or blood vessels of interest; and the blood vessels to be tested include coronary blood vessels, branch blood vessels originating from coronary blood vessels, vascular trees, single-vessel segments, and other vascular tissues at any individual positions; The blood vessel model includes at least one of the geometric model and the blood flow model, and alternative terms for the blood vessel model can also be a lumen model, a fluid flow model, etc., which can reflect the shape of the individual blood vessel to be examined and the fluid flow in the blood vessel. further, the blood vessel model includes the length, diameter, bending angle of the blood vessel to be inspected, the existence of branch blood vessels in the blood vessel to be inspected, the angle of the branch blood vessel, the number of branch blood vessels, etc. and the blood vessel to be inspected the data related to the geometry.

In this embodiment, the alternative term of the lumen morphology model can also be a cross-sectional morphology model, and the lumen morphology model includes the presence or absence of a plaque, the position of the plaque, the size of the plaque, and the plaque. The angle formed, the composition of the plaque and the change of the plaque composition, the shape of the plaque and the change of the shape of the plaque; further the establishment of the lumen morphology model includes the following steps:
S1. Define the cross section at the proximal end to be inspected as a reference plane, and establish a center diameter line for obtaining the blood vessel model by using a centerline extraction method;
S2. Establish a coordinate system with the center point of the reference surface as the origin, segment the blood vessel to be examined in a direction perpendicular to the center diameter line, and project the inner and outer edges of each cross section in the coordinate system to obtain the plane geometric image of the lumen shape of the blood vessel to be inspected at each position, and the establishment of the lumen shape model is completed.

In the present invention, the plane geometric image of the lumen shape at each position needs to use the coordinate system established in step S2 as a reference to clarify the position of the plaque on each lumen section to facilitate subsequent fitting of the lumen shape model.

It should be noted that in the process of establishing the lumen morphology model, when the anatomical data is acquired by CT, OCT, IVUS and other detection methods, the lumen morphology model can be directly acquired through the blood vessel model. It is only necessary to ensure that the origin and coordinate directions of each lumen morphology model are consistent; when the anatomical data is acquired by X-ray and other detection means, since the blood vessel model is a three-dimensional model extending along the direction of blood flow, Then, when the lumen shape model is established through the blood vessel model, coordinate conversion of the blood vessel model is required to accurately reflect the cross-sectional shape of each section.

The processor is further configured to determine the blood vessel pressure difference between any two positions of the blood vessel to be inspected based on the preset shape difference function through the lumen shape model and the blood vessel model. Wherein, the morphological difference function is obtained by fitting and establishing the lumen morphology model, and is used to represent the function of the change of the lumen morphology at different positions of the blood vessel to be examined with the change of the distance x from the position to the reference point; and The morphological difference function includes a difference function related to the area, volume, edge position, and edge morphology of the blood vessel to be inspected, which can reflect the morphological difference between any two positions of the blood vessel to be inspected, and the difference function can be directly/indirectly obtained through the lumen morphology model.

In other embodiments, the anatomical data may also be defined as anatomical data and other parameters that can be directly and/or indirectly acquired from the image acquisition device and can reflect the shape of the lumen.

That is, in another context, the processor, the blood vessel to be examined, the anatomical data, the luminal shape model, and the blood vessel model may be different names with the same meaning.

The scale is that the scale is the distance between two adjacent cross-sections; the different scales include a first scale, a second scale, . . . , an n-th scale;

The first-scale morphological difference function ƒ₁(x) is used to detect the geometric morphological difference between two adjacent cross-sectional morphological models caused by the first type of lesion feature;

The second-scale morphological difference function ƒ₂(x) is used to detect the geometric morphological difference between two adjacent cross-sectional morphological models caused by the second type of lesion feature;

The n-th scale morphological difference function ƒₙ(x) is used to detect the geometric morphological difference between two adjacent cross-sectional morphological models caused by the n-th lesion feature.

Further, the method of establishing the blood vessel model in the present invention is basically the same as the method of establishing the blood flow model and the geometric model. The only difference is that the blood vessel model can simultaneously include the shape and Blood flow information, so in this embodiment, the specific method of establishing the blood vessel model will not be repeated here.

Of course, the factors affecting the vascular pressure difference in this device include medical history information and/or physiological parameters; the medical history information includes circulatory system diseases, respiratory diseases, neurological diseases, and other diseases that affect blood flow speed or blood viscosity. One or more of bone disease, digestive system disease, metabolic disease, tumor disease and family medical history; the physiological parameters include one or more of directly obtainable physiological information such as age, gender, blood pressure and body mass index.

Further, in the present invention, the processor can also be used to run the following formula to calculate the vascular pressure difference ΔP:

$$Δ P = ( {c}_{1} V + {c}_{2} {V}^{2} + … + {c}_{m} {V}^{m} ) * [ {α}_{1} * \int {f}_{1} ( x ) d x + {α}_{2} * \int {f}_{2} ( x ) dx + … + {α}_{n} * \int {f}_{n} ( x ) d x ]$$

Among them, V is the blood flow velocity, which is obtained directly/indirectly through the blood flow model; c₁, c₂, . . . , cₘ respectively denote the parameter coefficients of blood flow velocity V, and the parameter coefficients include blood viscosity influencing factors, blood turbulence influencing factors, and viscosity coefficients; further, m is greater than or equal to 1 The natural numbers denote the influence of different parameter coefficients on the blood flow velocity V to correct the pressure difference value ΔP to ensure the accuracy of the calculation of the blood vessel pressure difference ΔP. Preferably, in the present invention, the value of m is 2, and when m is 2, c₁ is a parameter coefficient caused by blood flow friction, and c₂ is a parameter coefficient caused by blood turbulence.

The α₁, α₂, . . . , αₙ are the weighting coefficients of the morphological difference functions ƒ₁(x), ƒ₂(x), . . . , ƒₙ(x) of the vascular lumen at different scales, where n is the scale greater than or equal to 1. Further, the increase of the weighting coefficient can further modify the shape difference function f(x) to ensure the accuracy of the shape difference fitting calculation between the two cross sections.

The present invention also provides another method for obtaining vascular pressure difference, the method comprising:
Receiving anatomical data of the blood vessel, and obtaining a geometric model of the target blood vessel according to the anatomical data;
Preprocessing the geometric model to establish a cross-sectional shape model of the target blood vessel at various positions between the proximal end and the distal end;
Using the proximal end point of the target blood vessel as a reference point, the cross-sectional morphological model at different scales is fitted to calculate the morphological difference function f(x) of the target vessel lumen, and the scale is the calculated morphological difference function f(x) is the distance between two adjacent cross sections;
At this time, the pressure difference value ΔP at any two positions of the target blood vessel, the calculation formula of the ΔP at different scales is:

$$Δ P = k * [ {α}_{1} * \int {f}_{1} ( x ) d x + {α}_{2} * \int {f}_{2} ( x ) dx + … + {α}_{n} * \int {f}_{n} ( x ) d x ]$$

Among them, k is a correction parameter, and k is a constant greater than or equal to 1;
The α₁, α₂, . . . , αₙ are the weighting coefficients of the morphological difference functions ƒ₁(x), ƒ₂(x), . . . , ƒₙ(x) of the vascular lumen at different scales;

Preferably, the different scales include a first scale, a second scale, . . . , an n-th scale;

The first-scale morphological difference function ƒ₁(x) is used to detect the geometric morphological difference between two adjacent cross-sectional morphological models caused by the first type of lesion feature;

The second-scale morphological difference function ƒ₂(x) is used to detect the geometric morphological difference between two adjacent cross-sectional morphological models caused by the second type of lesion feature;

The n-th scale morphological difference function ƒₙ(x) is used to detect the geometric morphological difference corresponding to two adjacent cross-sectional morphological models caused by the n-th lesion feature; wherein, the n is a natural number greater than or equal to 1.

Further, the correction parameter k is a value obtained directly/indirectly based on individual information, that is, in the present invention, the correction parameter k is data obtained directly/indirectly through estimation or testing equipment, and the correction parameter k may be related to the individual Specific information or general information.

The morphological difference function ƒ(x) is used to represent the cross-sectional morphological change at different positions of the target blood vessel as a function of the distance x from the position to the reference point. The acquisition of the morphological difference function ƒ(x) includes:
Based on the cross-sectional morphological model, establish the shape function of each cross-section;
Fit the morphological functions of two adjacent cross sections, and obtain the difference change function of two adjacent cross sections at different scales;
Taking the proximal end of the target vessel as the reference point, obtain the rate of change of the lumen shape with the distance x from the reference point according to the difference change function, and normalize the position parameters of the target vessel from the proximal end to the distal end to obtain the shape difference function f(x) finally.

The morphological function includes an area function, a diameter function, or an edge distance function, that is, in the present invention, the difference between two adjacent cross sections at different scales can be obtained by fitting between the cross-sectional area, diameter, or edge distance function. Change function; further, the change rate of the lumen shape with the distance x from the reference point is obtained through the difference change function, and the shape difference function f(x) is obtained. That is, the morphological difference function f(x) is a function related to the change in the cross-sectional area of the two cross-sections of the target blood vessel, the diameter change at each position, or the edge distance change at each position.

Further, the cross-sectional morphological model includes plaque information at each cross-sectional position, wherein the plaque information is the lesion information of the target blood vessel, and the cross-sectional morphological model is in the process of establishing The information also needs to include the presence or absence of plaques, the location of the plaque, the size of the plaque, the angle at which the plaque is formed, the composition of the plaque and the change in plaque composition, the shape of the plaque and the change in the shape of the plaque, and In this embodiment, the establishment of the cross-sectional morphological model includes the following steps:
S1. Define the cross-section at the proximal end of the target blood vessel as a reference plane, extract the centerline of the target blood vessel by a centerline extraction method, and establish a center diameter line for acquiring the geometric model;
S2. Establish a coordinate system with the center point of the reference surface as the origin, segment the target blood vessel in a direction perpendicular to the center diameter line, and project the inner and outer edges of each cross section in the coordinate system to obtain the target plane geometric image of the cross-section of the lumen at each position of the blood vessel, and the establishment of the cross-sectional morphological model is completed.

Among them, the plane geometric image of the lumen cross-section at each position needs to be referenced to the coordinate system established in step S2. This setting can clarify the position of the plaque on each cross-section, so as to facilitate the subsequent modeling of the cross-sectional morphological model. Together, to further clarify the influence of different plaque shapes on the vascular pressure difference.

It should be pointed out that the devices and functional modules in this specification are merely illustrative of the basic structure for realizing the technical solution, not the only structure.

In summary, the method for obtaining blood vessel pressure difference of the present invention establishes a cross-sectional shape model, obtains planar geometric images at various cross-sectional positions of the target blood vessel, and establishes the shape by fitting cross-sectional shape models at different positions The difference function introduces the concept of cross-sectional shape during the calculation of the vascular pressure difference, and comprehensively considers the influence of the position and shape of the plaque in the lumen on the calculation of the vascular pressure difference; so that the vascular pressure difference is obtained by the present invention The vascular pressure difference calculated by the method is more accurate and can accurately reflect the pressure changes at both ends of the target blood vessel; it is guaranteed that the vascular pressure difference calculated by the method of the present invention is accurate and reliable when applied to the calculation of other blood flow characteristic values.

It is to be understood, however, that even though numerous characteristics and advantages of preferred and exemplary embodiments have been set out in the foregoing description, together with details of the structures and functions of the embodiments, the disclosure is illustrative only; and that changes may be made in detail within the principles of present disclosure to the full extent indicated by the broadest general meaning of the terms in which the appended claims are expressed.

What is claimed is:

1. A method for obtaining vascular pressure difference, performed by a device for obtaining vascular pressure difference, comprising the following steps:
receiving anatomical data of the blood vessel, and obtaining a geometric model of the target blood vessel based on the anatomical data;
obtaining a blood flow model of the target blood vessel combining the anatomical data with individual data;
preprocessing the geometric model to establish a cross-sectional shape model of the target blood vessel at various positions between the proximal end and the distal end;
wherein the establishment of the cross-sectional morphological model comprises:
S1. defining the cross-section at the proximal end point of the target blood vessel as a reference plane, and obtaining the center diameter of the geometric model through a centerline extraction and establishment method;
S2. establishing a coordinate system with the center point of the reference surface as the origin, dividing the target blood vessel in a direction perpendicular to the center diameter line, and projecting the inner and outer edges of each cross-section in the coordinate system to obtain the plane geometric image of the lumen cross-section at each position of the target blood vessel, thereby completing the establishment of the cross-sectional morphological model;
wherein the cross-sectional morphological model includes the presence or absence of plaques on each cross-section, the position of the plaque, the size of the plaque, and the angle formed by the plaque, the composition of the plaque and the change in the composition of the plaque, and the shape of the plaque and the change in the shape of the plaque;
wherein, taking the proximal end point of the target blood vessel as a reference point, the cross-sectional morphological model at different scales is fitted to calculate the morphological difference function f(x) of the target vessel lumen, and the scale for calculating the morphological difference function f(x) is the distance between two adjacent cross sections;
wherein the pressure difference value ΔP at any two positions of the target vessel is calculated, based on the morphological difference function f(x) of the target vessel lumen and the blood flow model.

2. The method for obtaining vascular pressure difference according to claim 1, wherein the blood vessels comprise coronary blood vessels, branch vessels derived from coronary blood vessels, blood vessel trees, and single blood vessel segments; and the individual data includes individual general Parameters and individual-specific parameters; the blood flow model includes at least the blood flow velocity V of the target vessel.

3. The method for obtaining vascular pressure difference according to claim 1, wherein the pressure difference value ΔP is calculated by calculating the morphological difference function f(x) of the target vessel lumen at different scales and the blood flow model of the target vessel, the calculation formula of the ΔP at different scales is:
$$Δ P = ( {c}_{1} V + c {V}^{2} + … + {c}_{m} {V}^{m} ) * [ {α}_{1} * \int {f}_{1} ( x ) d x + {α}_{2} * \int {f}_{2} ( x ) d x + … + {α}_{n} * \int {f}_{n} ( x ) d x ]$$
among them, V is the blood flow velocity, which is obtained directly/indirectly through the blood flow model;
c₁, c₂, . . . , cₘ respectively denote the parameter coefficients of blood flow velocity V;
α₁, α₂, . . . , αₙ are the weighting coefficients of the morphological difference functions ƒ₁(x), ƒ₂(x), . . . , ƒₙ(x) of the vascular lumen at different scales;
m is a natural number greater than or equal to 1;
n is a natural number whose scale is greater than or equal to 1;
the different scales include a first scale, a second scale, . . . , an n-th scale;
the first-scale morphological difference function ƒ₁(x) is utilized to detect the geometric morphological difference between two adjacent cross-sectional morphological models caused by the first type of lesion feature;
the second-scale morphological difference function ƒ₂(x) is utilized to detect the geometric morphological difference between two adjacent cross-sectional morphological models caused by the second type of lesion feature;
. . .
the n-th scale morphological difference function ƒₙ(x) is utilized to detect the geometric morphological difference between two adjacent cross-sectional morphological models caused by the n-th lesion feature; wherein, n is a natural number greater than or equal to 1.

4. The method for obtaining vascular pressure difference according to claim 1, wherein the morphological difference function f(x) is used to indicate that the cross-sectional morphological changes at different positions of the target blood vessel follow the distance x from the position to the reference point. Function of change;
the acquisition of the morphological difference function f(x) includes:
based on the cross-sectional morphological model, establishing the shape function of each cross-section; the shape function includes area function, diameter function and edge position function;
fitting the morphological functions of two adjacent cross sections, and obtaining the difference change function of two adjacent cross sections at different scales; and
taking the proximal end of the target vessel as the reference point, obtaining the rate of change of the lumen shape with the distance x from the reference point according to the difference change function, and normalize the position parameters of the target vessel from the proximal end to the distal end Processing to obtain the morphological difference function f(x).

5. The method for obtaining vascular pressure difference according to claim 2, wherein the obtaining of the blood flow model further comprises correcting the blood flow model through medical history information and/or physiological parameter information, and passing the corrected the blood flow model is obtained; the blood flow model includes a fixed blood flow model and a personalized blood flow model;
the personalized blood flow model includes a resting state blood flow model and a load state blood flow model; when the blood flow model is a resting state blood flow model, the blood flow velocity V can pass the speed at which intravascular fluid is filled Obtained by calculation; or calculated by the shape of the vascular tree;
the morphology of the vascular tree includes at least one or more of the area and volume of the vascular tree and the lumen diameter of the vascular segment in the vascular tree; when the blood flow velocity V is obtained by calculating the shape of the vascular tree, the geometric parameters also include one or more of the length, perfusion area, and branch angle of the vessel segment in the vessel tree.

6. The method for obtaining vascular pressure difference according to claim 2, wherein the blood flow velocity V includes the blood flow velocity of the target blood vessel in the maximum congestion state and the blood flow velocity in the resting state; or, the preprocessing of the geometric model includes the correction of the geometric model through medical history information and/or physiological parameter information.

7. A device for obtaining vascular pressure difference, comprising:
a data collector, which is used to obtain and store the geometric parameters of the target blood vessel in the anatomical model of the vascular system;
a pressure difference processor, which is used to establish a blood flow model of the target blood vessel, and a geometric model corresponding to the target blood vessel established based on the geometric parameters;
the pressure difference processor is further configured to correct the geometric model and/or blood flow model, and obtain a cross-sectional shape model and a blood vessel pressure difference calculation model based on the corrected geometric model and the blood flow model; wherein the cross-sectional morphological model includes the presence or absence of plaques on each cross-section, the position of the plaque, the size of the plaque, and the angle formed by the plaque, the composition of the plaque and the change in the composition of the plaque, and the shape of the plaque and the change in the shape of the plaque;
wherein the establishment of the cross-sectional morphological model comprises:
S1. defining the cross-section at the proximal end point of the target blood vessel as a reference plane, and obtaining the center diameter of the geometric model through a centerline extraction and establishment method;
S2. establishing a coordinate system with the center point of the reference surface as the origin, dividing the target blood vessel in a direction perpendicular to the center diameter line, and projecting the inner and outer edges of each cross-section in the coordinate system to obtain the plane geometric image of the lumen cross-section at each position of the target blood vessel, thereby completing the establishment of the cross-sectional morphological model;
wherein, at the same time, according to the blood vessel pressure difference calculation model and hemodynamics, the pressure difference value ΔP of the target blood vessel is obtained.

8. The device for obtaining vascular pressure difference according to claim 7, wherein the geometric model is obtained by measuring and calculating the image data of the anatomical model and fitting and calibrating; and the cross-sectional morphological model is obtained directly/indirectly from the geometric model.

9. The device for obtaining vascular pressure difference according to claim 7, wherein the geometric model obtained by the pressure difference processor includes at least one vascular tree, and the vascular tree includes at least a segment of aorta or at least a segment of aorta and multiple coronary arteries originating from the aorta; or the geometric model includes at least a single vessel segment.

10. The device for obtaining vascular pressure difference according to claim 7, wherein the apparatus for obtaining blood vessel pressure difference further comprises a speed collector, and the speed collector is used to obtain the blood flow speed of the target blood vessel, and the blood flow the speed is used to calculate the pressure difference value ΔP between the proximal end and the distal end of the target blood vessel;
wherein the speed collector includes a speed calculation module and a speed extraction module; the speed extraction module can directly collect the blood flow speed through the data collector, or directly extract the blood flow speed through the blood flow model; and
wherein the speed calculation module includes a speed conversion module and a speed measurement module. The blood flow speed can be obtained by converting the speed of fluid filling in blood vessels by the speed conversion module, and can also be obtained by converting the shape of the blood vessel tree in the geometric model by the speed. Obtained by calculation module.

11. A device for obtaining blood flow reserve score, comprising:
a data collector, which is used to obtain and store the geometric parameters of the target blood vessel in the anatomical model of the blood vessel device; and
a blood flow information processor, which is used to establish a blood flow model of the target blood vessel, and to establish a geometric model corresponding to the target blood vessel based on the geometric parameters;
wherein the blood flow information processor is also used to correct the geometric model and the blood flow model to obtain a cross-sectional shape model; wherein the cross-sectional morphological model includes the presence or absence of plaques on each cross-section, the position of the plaque, the size of the plaque, and the angle formed by the plaque, the composition of the plaque and the change in the composition of the plaque, and the shape of the plaque and the change in the shape of the plaque;
wherein, based on the cross-sectional morphological model and the blood flow model, a vascular pressure difference calculation model and the maximum blood flow velocity of the target vessel are obtained; and according to the vascular pressure difference calculation model and the maximum blood flow velocity, combined with hemodynamics, the blood flow reserve fraction FFR is calculated and obtained;
wherein the establishment of the cross-sectional morphological model comprises:
S1. define the cross section at the proximal end of the target blood vessel as a reference plane, and obtain the center diameter of the geometric model through a centerline extraction and establishment method;
S2. establish a coordinate system with the center point of the reference surface as the origin, segment the target blood vessel in a direction perpendicular to the center diameter line, and project the inner and outer edges of each cross section in the coordinate system to obtain the target, the plane geometric image of the cross-section of the lumen at each position of the blood vessel, the establishment of the cross-sectional morphological model is completed.

12. The device for obtaining blood flow reserve score according to claim 11, wherein the geometric model is obtained by measuring and calculating the image data of the anatomical model and fitting calibration; the cross-sectional morphological model is obtained by the geometric model is directly/converted to obtain;
when the image data received by the data collector is angiographic image data of the target blood vessel, the image data collected by the data collector is not less than two groups, and there is a collection angle between any two groups of the image data Difference, and the acquisition angle difference is not less than 20 degrees.

13. The device for obtaining blood flow reserve score according to claim 11, wherein the geometric model obtained by the blood flow information processor includes at least one vascular tree, and the vascular tree includes at least a segment of aorta or at least a segment of aorta. Arteries and multiple coronary arteries originating from the aorta; or the geometric model includes at least a single vessel segment;
the blood flow model established by the blood flow information processor includes a fixed blood flow model and a personalized blood flow model; the personalized blood flow model includes a resting blood flow model and a stress blood flow model;
when the blood flow model is a resting blood flow model, the maximum blood flow velocity can be obtained by calculating the speed of fluid filling in the blood vessel; or by calculating the shape of the vascular tree;
the shape of the vascular tree includes at least one or more of the area and volume of the vascular tree and the lumen diameter of the vascular segment in the vascular tree; when the maximum blood flow velocity is obtained by calculating the shape of the vascular tree, The geometric parameters also include one or more of the length, perfusion area, and branch angle of the vessel segment in the vessel tree.

14. The device for obtaining blood flow reserve score according to claim 11, wherein the device for obtaining blood flow reserve score further comprises a speed collector, which is used to obtain the maximum blood flow speed of the target blood vessel, and the maximum blood flow The flow velocity is used to calculate the first blood flow pressure Pa at the proximal end of the target blood vessel and the pressure difference value ΔP between the proximal end and the distal end of the target blood vessel.

15. A device for obtaining a patient's blood vessel pressure difference, which has a processor, wherein the processor is configured to make the device execute the following steps:
collecting the anatomical data of the patient's blood vessel to be examined;
establishing a blood vessel model of the patient's blood vessel to be examined according to the anatomical data;
based on the blood vessel model, further establishing a lumen morphology model at different scales;
wherein, according to the preset morphological difference function, the vascular pressure difference between any two positions of the blood vessel to be examined is determined based on the lumen morphological model and the blood vessel model;
wherein the morphological difference function is obtained by fitting and establishing the lumen morphological model, and is used to represent the function of the cross-sectional morphological changes at different positions of the target blood vessel when the distance x from the position to the reference point changes; and the morphological difference function includes a difference function related to the cross-sectional area or diameter or edge distance of the target blood vessel.

16. The device for obtaining a patient's blood vessel pressure difference according to claim 15, wherein the scale is the distance between two adjacent cross sections.

17. A method for obtaining vascular pressure difference, wherein the method includes:
receiving anatomical data of the blood vessel, and obtaining a geometric model of the target blood vessel according to the anatomical data;
preprocessing the geometric model to establish a cross-sectional shape model of the target blood vessel at various positions between the proximal end and the distal end;
using the proximal end point of the target blood vessel as a reference point, the cross-sectional morphological model at different scales is fitted to calculate the morphological difference function f(x) of the target vessel lumen, and the scale for calculating the morphological difference function f(x) is the distance between two adjacent cross sections;
wherein the calculation formula of the pressure difference value ΔP at any two positions of the target blood vessel at different scales is:
$$Δ P = k * [ {α}_{1} * \int {f}_{1} ( x ) d x + {α}_{2} * \int {f}_{2} ( x ) d x + … + {α}_{n} * \int {f}_{n} ( x ) d x ]$$
among them, k is a correction parameter, and k is a constant greater than or equal to 1;
α₁, α₂, . . . , αₙ are the weighting coefficients of the morphological difference functions ƒ₁(x), ƒ₂(x), . . . , ƒₙ(x) of the vascular lumen at different scales;
the different scales include a first scale, a second scale, . . . , an n-th scale;
the first-scale morphological difference function ƒ₁(x) is used to detect the geometric morphological difference between two adjacent cross-sectional morphological models caused by the first type of lesion feature;
the second-scale morphological difference function ƒ₂(x) is used to detect the geometric morphological difference between two adjacent cross-sectional morphological models caused by the second type of lesion feature;
. . .
the n-th scale morphological difference function ƒₙ(x) is used to detect the geometric morphological difference between two adjacent cross-sectional morphological models caused by the n-th lesion feature; wherein, n is a natural number greater than or equal to 1.

18. The method for obtaining vascular pressure difference according to claim 17, wherein the correction parameter k is a value obtained directly/indirectly based on individual information;
wherein the morphological difference function f(x) is used to represent the function of the cross-sectional morphological changes at different positions of the target blood vessel as the distance x from the position to the reference point changes.
